(19) United States
(12) Patent Application Publication
(10) Pub. No.: US 2015/0132346 A1
(43) Pub. Date: May 14, 2015

(54) MATRIX CONTAINING PIGMENT PARTICLES AND USE OF SAME IN COSMETIC COMPOSITIONS

(71) Applicants: Yun Shao, South Plainfield, NJ (US); Pascal Delrieu, Castanet (FR); David Schlossman, Short Hills, NJ (US); Frank Mazzella, Franklin Park, NJ (US)

(72) Inventors: Yun Shao, South Plainfield, NJ (US); Pascal Delrieu, Castanet (FR); David Schlossman, Short Hills, NJ (US); Frank Mazzella, Franklin Park, NJ (US)

(21) Appl. No.: 14/281,872

(22) Filed: May 19, 2014

Related U.S. Application Data
(63) Continuation-in-part of application No. 14/229,818, filed on Mar. 28, 2014.
Application No. 14/229,818 is a division of application No. 12/403,431, filed on Mar. 13, 2009, now abandoned.
Application No. 12/403,431 is a continuation-in-part of application No. 13/231,110, filed on Sep. 13, 2011.
Application No. 13/231,110 is a continuation-in-part of application No. 12/420,983, filed on Apr. 9, 2009, now abandoned.
(60) Provisional application No. 61/044,274, filed on Apr. 11, 2008.

(51) Int. Cl. A61K 8/81; A61Q 1/10; A61Q 19/00; A61K 8/02; A61K 8/19

(52) U.S. Cl. CPC A61K 8/8152; A61K 8/022; A61K 8/19; A61Q 19/00; A61Q 1/10
USPC 424/401; 514/782; 514/772.4; 514/772.3; 424/125

(57) ABSTRACT

A macroparticle pigment powder and methods of making the same. The macroparticle pigment powder comprises a plurality of macroparticle particles. The macroparticles are comprised of a matrix material that include pigment particles. In some embodiments the matrix material is polymeric in nature and selected from the group consisting of a natural latex, acrylics, polyacrylate, polymethacrylate, polyurethane, polyvinyl acetate, styrene-butadiene rubber, acrylonitrile butadiene styrene copolymer, and combinations thereof.

### CROSS REFERENCE TO RELATED APPLICATIONS

This application is a continuation in part of co-pending U.S. patent application Ser. No. 14/229,818 filed on Mar. 28, 2014, entitled POROUS AND/OR HOLLOW MATERIAL CONTAINING UV ATTENUATING NANOPARTICLES, METHOD OF PRODUCTION AND USE, which claims the benefit of Ser. No. 12/403,431 filed Mar. 13, 2009, entitled POROUS AND/OR HOLLOW MATERIAL CONTAINING UV ATTENUATING NANOPARTICLES, METHOD OF PRODUCTION AND USE, which claimed priority from U.S. Provisional Application No. 61/044,274 filed Apr. 11, 2008, entitled COMPOSITE POWDER ENCAPSULATING METAL OXIDE, THE MAKING AND ITS USE IN COSMETIC COMPOSITION, and a continuation in part of co-pending U.S. patent application Ser. No. 13/231,110 filed on Sep. 13, 2011, entitled MATRIX CONTAINING METAL OXIDE PARTICLES AND USE OF SAME IN COSMETIC COMPOSITIONS, entitled which claims the benefit of U.S. patent application Ser. No. 12/420,983 filed Apr. 9, 2009, entitled LARGE ULTRAVIOLET ATTENUATING PIGMENTS. This patent application makes reference to, claims priority to and claims benefit from the aforementioned applications and each of the applications is hereby incorporated herein by reference in its entirety.

### FIELD OF THE INVENTION

The present invention relates to pigments particularly useful for cosmetics, and is directed to a macroparticle matrix containing nanoparticle pigment particles, methods for forming the matrix, and compositions containing the same.

### BACKGROUND

Pigments are commonly used in industry as insoluble coloring agents in numerous colored or coloring compositions (paints, inks, and such make-up products as face powder, mascara and so forth). They are normally incorporated in these compositions in the particulate form or dispersed in bases or binders and applied to a surface such as a fingernail.

The development of new pigments is the subject of major efforts in, for example, the cosmetics industry. The cosmetics industry is looking more particularly for pigments which produce a superior visual effect as regards the intensity of the tints or colors, or the gloss, in order to incorporate them, for example, in make-up compositions applied to the skin, lips, nails or keratinous fibers, such as the eyelashes or eyebrows.

Mascaras are generally prepared in two types of formulations: aqueous mascaras referred to as cream mascaras, in the form of a dispersion of waxes in water, and anhydrous or low-water-content mascaras, referred to as water-resistant mascaras (also sometimes referred to as “waterproof”), in the form of dispersions of waxes in organic solvents.

However, the mascaras described above have the disadvantages of, among other things, being dry, having poor flexibility and/or having poor consistency due to uneven distribution.

There is thus a need to develop a cosmetic composition, in particular for making up the eyelashes, making it possible to obtain a smooth and homogeneous deposit on the eyelashes, while exhibiting a consistency that is easy to work after application, which has improved volumizing, curling and/or shine properties.

To improve the aesthetics of pigments, micro or nano particle pigments and dyes can be used in cosmetics. They are often sheer or transparent on the skin and aesthetically appealing and are extensively used nowadays. These micro grades typically have primary particle sizes of less than 100 nm and are referred to as nanoparticles.

Many other materials used in personal care products also contain so-called nanoparticles. For example, transparent oxides are used in color cosmetics. Carbon black (Black No. 2) is commonly used in mascara and eyeliners.

These micro grades typically have primary particle sizes of less than 100 nm when analyzed using TEM. Particles less than 100 nm are often referred to as nanoparticles. It has been found that such nanoparticle pigments result in an added degree of transparency in the cosmetic products into which they are incorporated.

However, there is concern that nanoparticles could penetrate the skin and cause harm to human health. Thus, these products in recent years have come under heightened scrutiny.

In view of the perceived health risk associated with nanoparticles, pigment producers have been long challenged to produce particles and/or composite powders with particles that are all or almost all larger than 100 nm, and preferably larger than 150 nm, as measured by TEM, yet still enable transparency of nanoparticles and desirable formulation characteristics of larger particles at the same time without undesirable or possibly questionable properties of either.

### SUMMARY

To address industry desires, the challenge is to create particles that are larger yet still have the desirable attributes such as good pigmentation that nanoparticles offer.

Accordingly the aim of the composition herein is to create an overall larger particle size while maintaining the desirable characteristics of micro particles.

In accordance with the invention, this has been accomplished by forming a pigmentation particulate product comprising a micro particles in a composite matrix material. The matrix material is one that is capable of forming, for example, a gel forming a solid particle when dried to entrap a plurality of pigment particles or a material exhibiting sufficient adhesion to bind the pigment particles without significantly interfering with the visual effect of said particles. The matrix material is preferably one that when dried is insoluble in the final product and preferably does not substantially swell in water or oil.

Preferable materials for the matrix include natural latex, acrylics, acrylate including, polyacrylate, polymethacrylate, polyurethane, polyvinyl acetate, styrene-butadiene rubber, acrylonitrile butadiene styrene copolymer, or combinations thereof. The matrix is formed, for example, by spraying a mixture of a liquid, such as water incorporating nanoparticles, and the matrix-forming material to form an aerosol-like mist suspended in air, for example warm air, causing the matrix-forming material to form a matrix, for example by drying, curing and/or polomerization. The result of such matrix formation is the transformation of the mixture into a fine powder which is recovered, and in accordance with an exemplary embodiment ground to a smaller particle size. Surprisingly, the resulting dried powder imparts substantially the same pigment power to a final cosmetic composition as a dispersion of the same micro pigment. Additionally, the matrix material is such that, after formation of the macroparticle and incorporation of the macroparticle into the final cosmetic composition, the microparticles cannot diffuse out of the resulting macroparticles.

In one aspect, a method for forming a macroparticle powder with colorants is disclosed. The method includes the steps of dispersing a pigment in water, adding a matrix material, spray drying and grinding to form a ground particulate composite matrix pigment for introducing color into a cosmetic product. In preferred embodiments, the matrix material is selected from the group consisting of a natural latex, aqueous emulsions or dispersions of acrylics, polyacrylate, polymethacrylate, polyurethane, polyvinyl acetate, styrene-butadiene rubber, acrylonitrile butadiene styrene copolymer, and combinations thereof to the pigment in water to form a mixture, and spray drying the mixture to obtain macroparticles.

The inventive macroparticles may be formulated into dispersions (available to cosmetic manufacturers to use as an ingredient in making cosmetics) and/or directly incorporated into cosmetic compositions during the manufacturing process, including but not limited to, pigments or suntan lotions, bronzers, other lotions, gels, hairsprays, mascara, foundation, and face powder.

A preferred embodiment of the present invention is a cosmetic composition having a treated pigment comprising a matrix entrapping carbon black powder as a pigment.

Carbon black has a deep black color (deeper than black iron oxide) and is often used in mascaras with iron oxides to deepen the color. Typically carbon black is milled into nanoparticles. While carbon black may provide superior visual qualities, it is often difficult to formulate due to the challenges associated with nanoparticle formulation. For example, the carbon black is very dusty and tends to stain because of its low density. More importantly, because of its very fine primary particle size, particle aggregation is severe and milling is typically necessary to develop the color strength of the carbon black.

### BRIEF DESCRIPTION OF DRAWING

FIG. 1 shows two Leneta cards comparing color intensity of mascara formulations using prior art dispersion and the present inventive matrix

### DETAILED DESCRIPTION

As used herein, the terms “microparticles,” “nanoparticles” and “micronized particles” are interchangeable and include a material having 5% or more of the nanoparticles, in which particles have a mean size less than about 100 nm, for example ranging from 50 to 150 nm.

The entrapped property of the pigment nanoparticles in the nanoparticle-particulate composite powder makes the powder especially attractive to formulators in the cosmetics industry, allowing for these powders to be used for a wide range of applications without undue concern regarding possible adverse effects due to the nanoparticles contacting skin or other surfaces to which it is applied. Thus, the formulator may freely incorporate the colorant nanoparticle-particulate composites of the present invention fabricated from substances such as metal oxides, dyes, and lakes into cosmetic compositions to meet an exceptionally diversified range of cosmetics requirements. In preferred embodiments, the use of carbon black with the matrix is particularly advantageous as discussed below.

The powder of the present invention provides all the benefits of using nanoparticles such as good transparency, good skin feel and other attributes, without placing the nanoparticles into unimpeded direct contact with the skin or other surface. The inventive macroparticle powder also may also be given an additional coating to achieve good dispersion stability and to improve its properties to make desirable cosmetics. Alternatively, the matrix may be formed of materials having a desired hydrophilic, hydrophobic or ampiphillic characteristic which is imparted to the inventive composite pigment powder.

The powder of the present invention may be formulated into a dispersion that is incorporated into a composition such as a cosmetic composition. The cosmetic composition may be a liquid or dry make-up such as foundation or pressed powder, lipstick, blush, eye shadow, or mascara. Additionally, the cosmetic composition may be anhydrous or an emulsion.

The nanoparticles may comprise inorganic pigments, dyes, and mixtures thereof. Suitable inorganic pigments may include, without limitation, zirconium oxide; iron oxides; aluminum oxide; chromium oxide; cerium oxide; manganese; clear plastics; high index of refraction glass; violet; ultramarines, composites of metal oxides or of a metal oxide and an inorganic salt and any other inorganic pigment powder useful in the cosmetic or other relevant arts.

Suitable dyes include lakes of calcium, barium, aluminum or zirconium salts of FD&C and D&C grades of Red No. 6, Red No. 7, Red 21, Red No. 27 and Yellow No. 5. Other suitable pigments include ferric blue, in particular carbon black. Other suitable pigments or dyes are known or will become apparent to those skilled in the art.

Particles used to make the inventive powder may comprise dermatologically acceptable pigments selected from the group consisting of inorganic pigments, organic pigments, organic lake pigments, pearlescent pigments, and mixtures thereof. When employed, the pigments are present in proportions depending on the color and the intensity of the color that it is intended to produce. The level of pigments in the solid portion of the composition may be from at least about 10-60% by weight, preferably, 30-50%. The pigments may be surface-treated with treatments that include, but are not limited to, silicones, perfluorinated compounds, lecithin, and amino acids.

The pearlescent pigments useful in the present invention include those selected from the group consisting of mica (or a similar plate-like substrate) coated with any of the following materials alone or in combination: titanium dioxide, bismuth oxychloride, iron oxides, ferric ferrocyanide, chromium oxide, chromium hydroxide, and any organic pigment of the above-mentioned type and mixtures thereof.

The phrase “primary particle size,” as used herein, refers to the diameter of a particle when the particles are substantially spherical or spheroidal and the width or the smallest dimension of a particle when the particles are acicular. Irregular shapes and agglomerations thereof include, but are not limited to, nodular, acicular-like, granular, ellipsoidal, hexagonal, prismatic, star-like, flakes, and Y-shaped. For pigment agents, the particle size is typically reported as the mean primary particle size as determined by electron microscopy. Frequently, the primary particles form aggregates due to inter-particle forces.

In another embodiment, these powders may include an organic coating that gives the colorant particles hydrophobic properties. The organic coating may be applied to the inorganic coating, if present, or directly to the colorant particles.

The hydrophobic coating agent may be, for example, a silicone, a silane, a metal soap, a titanate, an organic wax, and mixtures thereof. The hydrophobic coating may alternatively include a fatty acid, for example, a fatty acid containing 10 to 20 carbon atoms, such as lauric acid, stearic acid, isostearic acid, and salts of these fatty acids. The fatty acid may be isopropyl titanium triisostearate. With respect to the silicone, the hydrophobic coating may be a methicone, a dimethicone, their copolymers or mixtures thereof.

The invention is directed to a nanoparticle pigments in a macroparticle matrix. In addition to the toxicity issues previously mentioned nano-sized pigments tend to aggregate and agglomerate. This agglomeration makes the pigment less efficient. For pigments (i.e. color), the color intensity is lower per weight when agglomerated. Typically, the pigments are first dispersed to the smallest aggregates where size is controlled to optimize the best color per weight. Then, this pigment dispersion is added to the matrix material and the composite particle is formed by drying. As the pigment aggregates stay at a similar size, the resulting composite retains the desired color characteristics (trueness of share, intensity, consistency) within the matrix while avoiding the pitfalls associated with nanoparticles.

When using a light scattering size analyzer for measurement, the mean size of the composite particles in the inventive macroparticle powder ranges from about 0.2 microns to about 50 microns. Particles are under 100 nm are virtually eliminated.

Most importantly, in addition to mitigating the above noted weaknesses of nanoparticles, the inventive composite powder imparts enhanced skill feel. Further the matrix material is soft while some pigments have a tendency to have a rough-draggy feel. This is virtually eliminated when the inventive composite powder matrix is used in place of a simple pigment.

Very significantly, the use of the macroparticle matrix also benefits cosmetic manufacturers as it is easier to formulate with the inventive “large” micron-sized product, being, for example, easier to pour and easier to disperse than a sub-micron “nano” pigment.

Still yet another issue addressed by the invention is that nanopigments have a tendency to become airborne making the nanoparticles difficult to disperse. The inventive composite, in the cosmetic formulation environment, address this issue effectively.

Still yet another advantage of the invention is that nanoparticles in general also have a tendency to agglomerate. Color is a matter of aggregate size, and, therefore, of how well agglomerates are processed. With the present composites, there is less agglomeration resulting in a smoother product and more consistent color.

The macroparticle powder as described herein may contain a single type of colorant agent or combination of colorant agents. Furthermore, the agents in the macroparticle powder may also be combined with additional substances, such as, for example, photostabilizers, cosmetic oils and/or anti-oxidants.

The matrix material is one that is capable of forming, for example, a gel in soluble solids upon drying to entrap a plurality of particles of the Pigment agent or a material exhibiting sufficient adhesion to bind the Pigment agent particles without significantly interfering with the ultraviolet filtering ability of the Pigment agent particles or the transparency of the composition in the visible light region.

The matrix material is one that when dried or cured is insoluble and does not swell in water or oil. In preferred embodiments, the matrix material is selected from a natural latex, acrylics, aqueous emulsions or dispersions of acrylates including polyacrylate, polymethacrylate, polyurethane, polyvinyl acetate, styrene-butadiene rubber, acrylonitril butadiene styrene copolymer, or combinations thereof. The matrix is spray dried after being mixed with the colorant agent and a dried, fine powder is recovered. The resulting macroparticles are themselves a matrix having a plurality of the colorant agents embedded therein. This matrix is generally similar to cement, for example, where there is a primary material in which lumps of coarser material, as of an aggregate, are embedded. The matrix material is not an absorbent material, powder, sphere, microsphere, or the like.

The macroparticle powder made by the process discussed above typically comprises the colorant agent and matrix material present at a ratio (by dry weight) of about 5:1 to about 1:20 more preferably from about 2:1 to about 1:2.

Each macroparticle powder particle typically contains a plurality of colorant agent particles. It is also contemplated that various particle types and/or particles of various sizes may be combined in a single macroparticle powder particle.

Macroparticle powders may be formed by any method capable of producing the macroparticle powder particles at the appropriate size. In preferred embodiments, the present invention is described in more detail by reference to spray drying to form the macroparticle powder. However, the present invention should not be considered limited to this process and other processes such as freeze drying, prilling, extrusion/spherization, emulsion/dispersion process and precipitation may also be used. The dried, fine powder resulting after macroparticle formation may have a particle size of about 0.2-100 micrometers.

It is also contemplated that after macroparticle formation, the macroparticles may be screened, milled, or reduced in size by other processes to assure proper particle size to meet a required size specification.

Spray drying is a particle processing technology that transforms a liquid feed stock into a powder product by first spraying the feed stock to create droplets, and then evaporating the feed stock liquid through the use of a heated drying medium, typically air. The liquid feed stock can take the form of a solution, suspension, liquid-paste or emulsion, and should be pumpable and capable of droplet formation. The feed stock composition in accordance with the present invention comprises the colorant agent, the matrix material and a dispersion media, such as, for example, water or organic solvents.

The macroparticle powder prepared in accordance with the present invention may be formulated into cosmetic compositions, pigment compositions, or other compositions. The macroparticle powder may be incorporated into the finished compositions with a concentration from about 1 to about 80% by weight, more preferably from about 2-20%, and most preferably from about 3 to about 10% by weight. The finished compositions may be in the form of suntan lotions, bronzers, other lotions, gels, hairsprays, mascara, foundation, face powder, aerosol foam creams or emulsions, and so forth.

The cosmetic compositions containing the macroparticle powders above may be formulated in various forms by conventional methods. Although the forms are not particularly limited, the cosmetics may be formulated as various makeup products as noted above and including lotions, emulsions, creams, ointments, aerosol cosmetics, powdery foundations, powdery eye shadows, emulsifying foundation creams, lipsticks, hair care preparations, and skin cleansers.

The cosmetically acceptable medium in which the pigment of the invention is dispersed advantageously comprises one or more cosmetic excipients advantageously chosen from dispersing agents, preservatives, antioxidants, fragrances, rheology agents, texturing agents, polymers or surfactants.

The polymers can advantageously be chosen for their ability to form a film at the time of their application and, in this case, are advantageously combined with plasticizing agents capable of improving the mechanical properties (elasticity, strength) of the film formed at the time of application of the composition comprising the pigment of the invention.

The medium can be an aqueous or hydrophilic medium or else can be formed of a fatty phase formed of lipophilic or liposoluble compounds.

In a preferred embodiment, the invention is a cosmetic composition comprising a matrix entrapping carbon black powder. According to a preferred embodiment, the particles are dispersed in a translucent or transparent cosmetic base as such a base makes it possible to optimally display the visual effect provided by the particles.

The term “carbon black” includes carbon black in the pure state or any black pigment essentially composed of carbon black, i.e. at least 90% and better still at least 95% by weight of carbon black or D & C Black No. 2 that is certified by the FDA for Drug and Cosmetic use.

The term “primary pigment” is also used with reference to the absence of treatment of this pigment.

The entrapment of the pigment in a matrix makes it possible to significantly increase the final particle size of the pigment of the invention, which makes the use thereof risk-free in the above-mentioned compositions.

The carbon black is generally incorporated in a proportion of between about 5 to about 60% by weight, preferably 23-43%, and more preferably 30-35% by weight in the case of carbon black.

The particles of pigment of the invention can be used as coloring fillers in various cosmetic compositions. Typically, the pigment of the invention is dispersed in a medium suitable for the preparation of colored and/or coloring compositions. The pigment according to the invention exhibits an excellent ability to be dispersed (dispersability) in pulverulent, liquid, semi-liquid, or pasty media.

The pigment of the invention when dispersed in such a medium produces, at the time of the application of the composition, a deeper black, in particular when this composition forms a clear, dry film.

The inventive micron-sized powder provides mechanical resistance, prevents aggregates from agglomerating, and gives improved tactile properties. The aggregates dispersed in the matrix give deep color when manufactured as agglomeration of particles is avoided. The particle size and composition provides a superior feel.

According to a preferred use, the cosmetic composition comprises from 1 to 99% by weight, advantageously from 1 to 80% by weight and more preferably from 3 to 70% by weight of the pigment of the invention, with respect to the total weight of the composition, the balance being composed of the cosmetically acceptable medium.

The medium in which the pigment of the invention is dispersed can also comprise an immiscible phase and can then be provided in the form of a water-in-oil or oil-in-water emulsion.

Depending on the composition under consideration, the medium can be solid and in particular pulverulent, pasty, liquid or semi-liquid.

As explained above, the cosmetically acceptable medium (also referred to as a “base” in the present patent application) is advantageously transparent or translucent and more advantageously transparent and clear.

According to a preferred use of the invention, the make-up composition is a mascara that, once applied to a hair, produces a visually deeper black color and a greater gloss.

The present inventive composite matrix is particularly advantageous when used for mascara. In addition, the idea of such formulations is to deposit black pigment at the surface of the eye-lashes, but there are other valued effects, in particular “volumizing”, i.e. making the eye-lash look thicker. This is achieved by using a resin, but also by adding particles that will stick on the surface of the eye-lash and allow more resin to stick to it. thus, “pay-off” (defined as the weight of mascara left on the eye-lashes after brushing the formula) is an important value. The higher the pay-off, the more mascara stays on the eye-lash, the darker the eye-lash appears, the thicker is looks.

The composite allows manufacturer to increase color intensity by using carbon black nanoparticles for a deeper black.

The mascara is advantageously formed of a dispersion of particles of the pigment of the invention in a transparent film-forming base.

The present invention is described in more detail by the following non-limiting examples.

### Example 1

### Composite with Carbon Black

Five (5) kg of a carbon black aqueous slurry (20 wt %) thereof (available as W20CB from Kobo Products, Inc.) was diluted in 10 kg of water. 2.2 kg of a 50% kaolin aqueous slurry was added to the diluted carbon black slurry. Finally, 1.6 kg of an acrylate emulsion with a 50% resin content (available as Daitosol 5000AD from Kobo Products, Inc.) was added to the dispersed carbon black with mixing. Thorough mixing was performed in a jacketed tank to ensure a uniform dispersion. The resulting mixture was then spray dried using a conventional spray drying apparatus at 375° C. A fine powder of about 17 microns in average size was obtained.

### Example 2

### Mascara Formulation

Two mascara formulations were made using the same basis composition (see Table 1).

TABLE 1

| Part | Ingredient | Formula A | Formula B |
|---|---|---|---|
| 1 | Beeswax White SP 422 | 8.0 | 8.0 |
| | CELLULOBEADS D-10 (Volumizer - | 5.0 | — |
| | 10 μm cellulose microsph [symbol] | | |
| | Ozokerite Wax White SP 1020 | 5.0 | 5.0 |
| | Carnauba Wax SP 63 | 3.0 | 3.0 |
| | Dermofat 4919 | 3.0 | 3.0 |
| | KOBOGUARD ® 5400 SQ | 2.0 | 2.0 |
| | Microcrystalline Wax SP-89 | 2.0 | 2.0 |
| | Liposorb ® SQO | 1.0 | 1.0 |
| | Propyl Paraben NF | 0.1 | 0.1 |
| 2 | Deionized Water | 31.7 | 37.6 |
| | Butylene Glycol | — | 2.0 |
| | Iron Oxide dispersion (W60BBNFAP) | 13.0 | 13.0 |
| | Triethanolamine | 1.0 | 1.0 |
| | Hydroxyethylcellulose | 0.3 | 0.3 |
| | Methylparaben | 0.2 | 0.2 |
| 3 | DAITOSOL 5000SJ | 10.5 | 10.5 |
| | Deionized Water | 6.0 | 6.0 |
| | Phenoxyethanol (And) Caprylyl Glycol | 0.6 | 0.6 |
| | (And) Potassium Sorbate (And) Water | | |
| | (And) Hexylene Glycol | | |
| | Imidazolidinyl Urea | 0.2 | 0.2 |
| 4 | Carbon Black dispersion (WBG20CB) | 7.5 | — |
| | Composite ACCB-33 (17 μm) | — | 4.6 |
| | Carbon Black content | 1.5% | 1.5% |

[symbol]

indicates data missing or illegible when filed

The mascara formulation steps are as follows:

### Process for Formula A

1. Combine the ingredients in Part 1 with the exception of the Cellulobeads. Heat and stir Part 1 to 75-80° C. until clear and then add the Cellulobeads.
2. Combine the ingredients in Part 2 with the exception of the Hydroxyethylcellulose and Iron oxide. Heat and stir Part 2 to 40-45° C. until clear and then add the Hydroxyethylcellulose.
3. Continue heating and stirring Part 2 to 60-65° C. and add the W60BBNFAP.
4. Homogenize Part 2 for 15 minutes. Continue heating and stirring Part 2 to 75-80° C.
5. Add Part 1 to Part 2 and begin air cooling with stirring.
6. At 65° C. add the pre-neutralized Daitosol 5000SJ to the batch.
7. Add the Imidazolidinyl urea to the deionized water in Part 3 and add it to the batch at 45° C.
8. Add the Carbon black dispersion to the batch and homogenize for 10 minutes. Continue stirring and force cool to 30° C. Fill mascara units.

Process for Formula B
1. Combine the ingredients in Part 1. Heat and stir Part 1 to 75-80° C. until clear.
2. Combine the ingredients in Part 2 with the exception of the Hydroxyethylcellulose and Iron oxide. Heat and stir Part 2 to 40-45° C. until clear and then add the Hydroxyethylcellulose.
3. Continue heating and stirring Part 2 to 60-65° C. and add the W60BBNFAP.
4. Homogenize Part 2 for 15 minutes. Continue heating and stirring Part 2 to 75-80° C.
5. Add Part 1 to Part 2 and begin air cooling with stirring.
6. At 65° C. add the pre-neutralized Daitosol 5000SJ to the batch.
7. Add the Imidazolidinyl urea to the deionized water in Part 3 and add it to the batch at 45° C.
8. Add the Carbon black composite to the batch and homogenize for 10 minutes. Continue stirring and force cool to 30° C. Fill mascara units.

Formula A, serving as the control, had 5% of 10 micron Cellulose microspheres (available as Cellulo Bead D10 from Kobo Products, Inc.) and 7.5% of a dispersion of carbon black in water and butylene glycol that has carbon black present as 20% by dry weight thereof (available as WBG20CB from Kobo Products, Inc.).

Formula B has no cellulose microsphere or carbon black dispersion; both ingredients were replaced with 4.6% Carbon Black Composite prepared as Example 1. Both formulas have the same percentage of carbon black (1.5%).

### Comparison

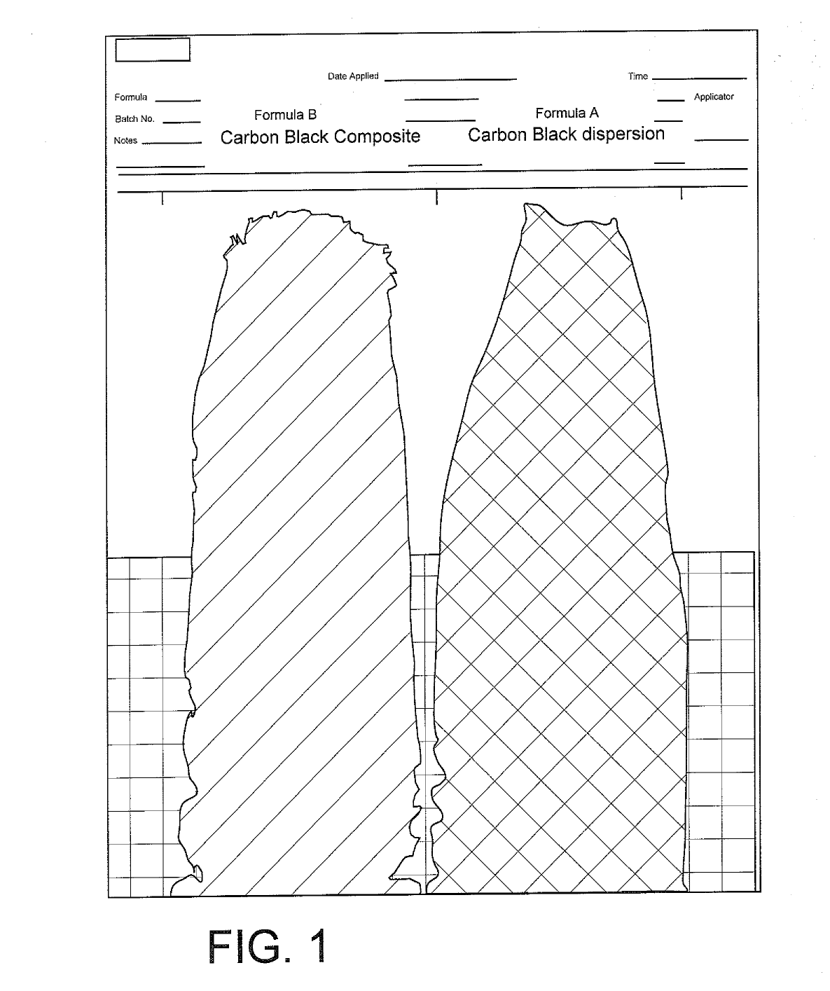

The two formulas were then compared for color intensity by comparing drawdowns on a Leneta cards (FIG. 1). Finally, the mascara formulas were applied with a brush to false eyelashes and eyelashes were weighed to measure the “pay-off” of the formulas as the amount of mascara deposited on the eyelashes. The two formulas were found to be of similar intensity, and to give similar pay-off. It shows that the Carbon Black composite as prepared in Example 1 gives similar results and can replace both a carbon black dispersion for color intensity and microsphere for pay-off in one ingredient, while not being considered a nano-sized ingredient.

### Example 3

### Iron Oxide Composite

While carbon black is a preferred pigment, other pigments can be used in the inventive composite, such as iron oxide. 4.5 kg of a nano-sized iron oxide aqueous slurry (35 wt %)(available as W35RTO from Kobo Products, Inc.) was dispersed in 7.5 kg of water. 3 kg of a 50% kaolin aqueous slurry was then added to the iron oxide suspension. Finally, 3 kg of an acrylate emulsion that is 50% resin by dry weight (available as Daitosol 5000AD from Kobo Products, Inc.) was added to the dispersed iron oxide with mixing. Thorough mixing was performed in a jacketed tank to ensure a uniform dispersion. The resulting mixture was then spray dried using a conventional spray drying apparatus at 375° C. A fine powder of about 22 microns in average size was obtained. After spray drying the particles, the particles may be recovered, dried and then milled or ground into about 9 microns

Although this pigment produces the most noteworthy visual effect when it is incorporated in a translucent or transparent base, such as a mascara or a lipstick, it is also possible to incorporate it in compositions for which the base is rendered opaque by the presence of cosmetic excipients. It is thus possible to prepare numerous compositions, in particular cosmetic compositions for coloring the skin, lips, nails, the hair or other keratinous fibers.

While illustrative embodiments have been described above, it is, of course, understood that various modifications will be apparent to those of ordinary skill in the art. Many such modifications are contemplated as being within the spirit and scope of the following claims.

1. A macroparticle colorant powder comprising: a plurality of macroparticle particles, the macroparticle particles comprising pigment particles in a matrix material selected from the group consisting of a natural latex, acrylic, polyacrylate, polymethacrylate, polyurethane, polyvinyl acetate, styrene-butadiene rubber, acrylonitrile butadiene styrene copolymer, and combinations thereof.

2. The macroparticle pigment powder of claim 1, wherein said matrix material is transparent or translucent.

3. The macroparticle pigment powder of claim 1, wherein, said macroparticle particles have an mean particle size of from about 0.2 micrometers to about 100 micrometers, and said pigment particles have an mean primary particle size of less than 100 nm.

4. The macroparticle pigment powder of claim 1, wherein the pigment particles comprise inorganic pigment particles.

5. The macroparticle pigment powder of claim 4, wherein the pigment particles have an mean particle size of less than 100 nm.

6. The macroparticle pigment powder of claim 7, wherein the pigment particles comprise carbon black.

7. The macroparticle pigment powder of claim 1, wherein the macroparticle particles comprise a ratio of pigment particles to matrix material of about 5:1 to about 1:20 by dry weight.

8. The macroparticle pigment powder of claim 1 wherein the macroparticle particles comprise a ratio of pigment particles to matrix material of about 2:1 to about 1:2 by dry weight.

9. A cosmetic composition incorporating the macroparticle pigment powder of claim 1.

10. A method for forming a macroparticle powder comprising:
dispersing a pigment in water; adding a matrix material selected from the group consisting of a natural latex, aqueous emulsions or dispersion of acrylics, polyacrylate, polymethacrylate, polyurethane, polyvinyl acetate, styrene-butadiene rubber, acrylonitrile butadiene styrene copolymer, and combinations thereof to the pigment in water to form a mixture; and drying the mixture to obtain macroparticles.

11. The method of claim 10, wherein the macroparticles have an average particle size of about 0.2-100 micrometers.

12. The method of claim 11, wherein the pigment has a mean particle size of less than 100 nm.

13. The method of claim 10, wherein drying includes spray drying.

14. The method of claim 10, wherein the pigment agent to matrix material comprise a ratio of about 5:1 to about 1:20 by dry weight.

15. The method of claim 10, wherein the pigment agent to matrix material comprise a ratio of about 2:1 to about 1:2 by dry weight.

16. A method of forming a pigmented material, comprising forming a composite pigment powder, said composite pigment powder being formed by:
(a) dispersing a visible color imparting pigment in a carrier liquid to form a carrier liquid with entrained pigment;
(b) adding a matrix-forming material to said carrier liquid with entrained pigment, said matrix-forming material being adapted to form a solid matrix;
(c) mixing said matrix-forming material and said carrier liquid to form a mixture; and
(d) drying the mixture to obtain macroparticles comprising said pigment incorporated in a matrix material.

17. A method as in claim 16, wherein said drying of the mixture to obtain macroparticles is done by dispersing said mixture in air or gas and evaporating volatile components of said mixture to form said matrix forming material into a solid matrix incorporating said pigment.

18. A method as in claim 16, wherein said drying of the mixture to obtain macroparticles is done by dispersing said mixture in air or gas and evaporating volatile components of said mixture to form said matrix forming material into a solid polymeric matrix incorporating said pigment.

19. A method as in claim 16, wherein said drying of the mixture to obtain macroparticles is done by dispersing said mixture in air or gas and evaporating volatile components of said mixture to form said matrix forming material into solid polymeric matrix powder incorporating said pigment and grinding said powder into non-spherical particles.

20. A method as in claim 19, wherein said matrix forming material is selected from the group consisting of a natural latex, aqueous emulsions or dispersion of acrylic, polyacrylate, polymethacrylate, polyurethane, polyvinyl acetate, styrene-butadiene rubber, acrylonitrile
